(19) United States
(12) Patent Application Publication
(10) Pub. No.: US 2013/0034530 A1
(43) Pub. Date: Feb. 7, 2013

(54) Dietary Supplement Cognitive Support System

(71) Applicant: David Robert Fantz, Denver, CO (US)

(72) Inventor: David Robert Fantz, Denver, CO (US)

(73) Assignee: David R. Fantz, Denver, CO (US)

(21) Appl. No.: 13/456,287

(22) Filed: Apr. 26, 2012

Related U.S. Application Data
(60) Provisional application No. 61/480,372, filed on Apr. 29, 2011.

(51) Int. Cl. A61K 33/30; A61P 3/02; A61P 25/00; A61K 38/54

(52) U.S. Cl. 424/94.2; 424/643

(57) ABSTRACT

The present invention relates to a nutritional supplement composition, comprising a therapeutically effective amounts of Vitamin C, Vitamin D3, Thiamin, Riboflavin, Niacin, Vitamin B6, Folic acid, Vitamin B12, Pantothenic acid, Calcium, Magnesium, Zinc, Chromium, Sugar, Protein, Acetyl-L-Carnitine, Dimethylaminoethanol complex, Phosphatidylserine complex, L-Glutamine, N-Acetyl-L-Tyrosine, L-Phenylalanine, Taurine, Methionine, Valine, Isoleucine, 5 Hydroxytryptophan, L-Taurine, N-Acetyl-Tyrosine, N-Acetyl-L-Cysteine, Alpha Lipoic Acid, Alpha Glycerylphosphoricholine complex, Bacopa Monnieri extract, Gingko Biloba extract, Passion flower, Lemon Balm, Gotu Kola, Ashwagandha, Choline Bitartrate complex, Panax Ginseng extract, Turmeric, Organic freeze dried fruit juice blends (concord grape, red raspberry, pineapple, cranberry, acai, pomegranate, acerola cherry, bilberry, lingonberry, black currant, aronia, sour cherry, black raspberry), Organic freeze dried greens blends (barley grass, broccoli, beet, carrot, alfalfa, oat), and Protein digestive enzyme blends (Protease 4.5, peptidase, bromelain, protease 6.0, protease 3.0, L planatrum, B bifidum) in a mixture to provide optimal cognitive function.

### FIELD OF THE INVENTION

The present invention relates generally to the field of nutrition, and more specifically to food and vitamin preparations comprising therapeutic levels of vitamins, and micronutrients suitable for human consumption.

### DESCRIPTION

### BACKGROUND OF THE INVENTION

Vitamin mineral, and amino acid supplement preparations are commonly administered as general nutritional supplements. Micronutrients are elements or compounds which are present in foods in small or trace amounts and include vitamins, minerals, or other elements, and compounds found in foods for which a Dietary Reference Intake (DRI) as established by the National Academy of Sciences has not yet been determined. The macronutrients consist of carbohydrates, fats, and proteins which supply nutrients and calories. Some elements such as calcium, sodium, potassium, chloride, and phosphorus are consumed in relatively large amounts, while many such as iron, iodine, and zinc are consumed in small amounts. Vitamins such as B12 and folic acid and minerals such as selenium, and cadmium are consumed in very small or trace amounts. In as much as the human body does not synthesize many compounds which are essential to the human body, these specific vitamins and minerals can be obtained from only two sources: food and supplements. The primary source of all nutrients is food. However, the majority of people do not meet the DRIs through consumption of the foods containing these essential compounds and elements. Thus vitamin and mineral supplementation has become a recognized method of meeting accepted health standards. Moreover, there is no current high quality regimen of dietary supplements that address the vitamin, mineral, amino acid and neural compound requirements of humans needed to support brain and cognitive function, behavior and mood.

### SUMMARY OF THE INVENTION

In one embodiment, the nutrition supplement composition of the present invention comprises, by way of example, a therapeutically effective amount of Vitamin C (from mineral ascorbates), Vitamin D3 (as cholecalciferol), Thiamin (B1) (as thiamin HCl), Riboflavin (B2), Niacin (as niacinamide), Vitamin B6 (as pyridoxine HCl), Folic acid, Vitamin B12 (as methylcobalamine), Pantothenic acid (as d-calcium pantothenate), Calcium (as calcium carbonate), Magnesium (as magnesium ascorbate), Zinc (as zinc ascorbate), Chromium (as chromium picolinate), Sugar (as glucose), Protein (from pea protein isolate), Acetyl-L-Carnitine, DMAE (Dimethylaminoethanol) complex, PS (Phosphatidylserine) complex, Proprietary blends (L-Glutamine, N-Acetyl-L-Tyrosine, L-Phenylalanine, Taurine, Methionine, Valine, Isoleucine, 5 HTP (Hydroxytryptophan), L-Taurine, N-Acetyl-Tyrosine, N-Acetyl-L-Cysteine, Alpha Lipoic Acid, Alpha GPC (Glycerylphosphoricholine) complex, Bacopa Monnieri (root) extract, Gingko Biloba (leaf) extract, Passion (flower), Lemon Balm (leaf), Gotu Kola (root), Ashwagandha (root), Choline Bitartrate complex, Panax Ginseng (root) extract, Turmeric), Organic freeze dried fruit juice blends (concord grape, red raspberry, pineapple, cranberry, acai, pomegranate, acerola cherry, bilberry, lingonberry, black currant, aronia, sour cherry, black raspberry), Organic freeze dried greens blends (barley (grass), broccoli (whole), beet (root), carrot (root), alfalfa (root), oat (grass)), and Protein digestive enzyme blends (Protease 4.5, peptidase, bromelain, protease 6.0, protease 3.0, L planatrum, B bifidum). In a preferred embodiment, the aforementioned composition is delivered through formats that are chewable, powder-based, or beverage. It also contains natural ingredients and is casein free, gluten free, and yeast free as well as having no additives, flavoring, or preservatives that are artificial. The nutrition supplement just described supports healthy neural development, cognitive function, concentration, memory, alertness, learning, and mood and behavioral stability which are all are rooted in biological processes that are supported by the essential nutrients provided by the current invention.

### DETAILED DESCRIPTION OF THE PREFERRED EMBODIMENT

A composition for human or animal consumption for supplying and delivering high potency levels, when compared to other multivitamins or FDA recommended Daily Values, of: C, D, B₁, B₂, Niacin, B₆, Folic Acid, B₁₂, Pantothenic Acid, Zinc, and Chromium. Studies show that these vitamins and minerals are beneficial at higher levels than the FDA Daily Values.

There have been no established negative side effects of higher levels of the B vitamins except for the following: the possibility of toxicity with higher levels of choline in those with kidney or liver dysfunction; flushing of the skin with higher levels of niacin; and the potential for masking of symptoms of vitamin B₁₂ deficiency with higher levels of folic acid. As a consequence, the current invention can be, in one example, formulated in consideration of these potential adverse effects of the B complex by providing a more conservative dose for choline, B₁₂, and folic acid. Other vitamins and minerals as defined in the composition have been clinically proven to not yield negative side effects at levels contemplated in the embodiment.

Vitamin Bᵢ has been shown to improve cognition in both children and adults when taken at any level, with higher doses having a more beneficial effect. While there are studies that supplemented vitamin B₁ along with other B vitamins improves cognitive abilities, there are few for vitamin B₁ alone to determine an optimal dose for healthy humans. One study actually showed that a daily dose of 100 mg (6,666% DV) reduced fatigue and boosted energy. Although one embodiment of the current invention's dose is more conservative than this study, it has been postulated in this invention that increased levels of thiamin will have a similar positive effect on humans.

Similarly, vitamin B₂ is known to be important for a healthy brain and nervous system as well as to reduce stress, but there are few studies on vitamin B₂ supplementation alone to determine an optimal dose for healthy humans. A recent study on a 16 year old with a nervous system disorder showed that a daily dose of 100 mg (5,882% DV) of vitamin B₂ daily significantly improved cognition and motor function. Although the current invention's dose is more conservative than this study, it has been postulated in this invention that increased levels of thiamin will have a similar positive effect on humans.

Niacin is commonly taken at daily doses of 1.5 g to 2 g (7,500-10,000% DV) to treat high cholesterol levels in adults. High levels at a lower dosage of 45 mg (225% DV) have been shown to improve cognition and memory in elderly adults. Although the current invention's dose is more conservative than this study, it has been postulated in this invention that increased levels of thiamin will have a similar positive effect on all humans.

The importance of pantothenic acid for a healthy brain and body has been demonstrated in numerous studies where it was supplemented along with the other B vitamins, but there are limited studies to date relating to vitamin pantothenic acid alone which provide an accurate view of its effects and optimal dosage for healthy children. One study of pantothenic acid supplementation showed the benefit of boosting energy.

Although there are limited studies on the effects of vitamin B₆ in healthy humans, it is well known for its many positive effects in hyperactive and autistic children. Most scientific studies involved supplementation with both vitamin B₆ and magnesium together rather than vitamin B₆ alone. In these studies the effective doses varied from just a few milligrams based on body weight all the way to a daily dose of 100 mg (5,000% DV). One embodiment of the current invention contains a higher level of B₆, compared to the daily dose there is reason to believe that increased levels of B₆ will have a positive effect.

Many published medical reports document a folic acid deficiency in cases of mental disorders such as depression, bipolar disorder and mania. One embodiment of the current invention includes folic acid supplementation to support optimal brain function in healthy humans. Folic acid has been supplemented at very high levels in some studies. For example, children with the blood disease sickle cell anemia were administered a daily dose of 1 mg (2,500% DV) with no reported side effects.

Vitamin B₁₂ is known to be important for brain health and energy. One study reported a daily dose of 15 mcg (250% DV) of vitamin B₁₂ increased the speed of processing, recall, recognition, verbal ability in the brain; and provided increased energy levels. Another study reported that taking 1 mg (16,666% Daily Value) of vitamin B₁₂ a day decreased cognitive impairment. One embodiment of the current invention contains a higher level compared to 100% of the Daily Value as recommended by the National Academy of Sciences.

The antioxidant vitamin C is a very important therapeutic nutrient that yields researched benefits at high doses. However, while it is typically present near 100% of the Daily Value levels in multivitamins it is generally under-represented in terms of effective daily dosages in supplements. In one of the purposes of the current invention a supplement is envisioned that will contain higher levels to ensure adequate protection from stress and free radicals in order to contribute to healthy human brains and bodies. Compared to the 8 g of vitamin C given per 70 kg of body weight daily in a study, the levels contemplated in the embodiment of the current invention are less than the higher levels tolerated in some studies.

Vitamin D is believed by scientists to be under consumed in the United States. According to a study in the Archives of General Psychiatry, vitamin D deficiency can adversely impact the brain and bones, and further that people with low vitamin D levels were at heightened risk of depression. Another clinical study found that vitamin D lifts low moods.

Essential minerals play a vital role in keeping the brain healthy. Chromium supports the brain's resistance to the degenerative effects of elevated blood sugar levels. Calcium, magnesium and zinc support focus and concentration as researched in clinical studies.

Amino acids play essential roles in cognitive nutrition. They are confirmed to be critical given the tremendous cell turnover within the human brain overtime, and requirements for specific amino acids in the formation of new neural cells. Complete, natural profiles of amino acids in addition to amino acid isolates contribute to the health of grey matter and neurotransmitters like GABA. Complete amino acid profiles provide better amino acid absorption and utilization, and enzyme supplementation when proteins are consumed enable more efficient utilization.

There exists a need for a nutritional supplement for humans which supplies the right amount of the right micronutrients at the right time to assure adequate intake of micronutrients needed for disease prevention and protection against nutritional losses and deficiencies due to lifestyle factors and common inadequate dietary patterns as well as promoting mental health.

In one aspect of the current invention, it has been postulated that the combination of the aforementioned components into a supplement form suitable for human consumption, will have positive long-term effects on growth, development and longevity of brain functions. In yet another benefit of the current invention, the formulation when administered will support the brain's ability to thrive and avoid loss of function due to lack of proper nutrients.

Thus, in one embodiment of the current invention a combination of the aforementioned vitamins and supplements in a therapeutically effective amount is formulated and designed for consumption by adults. The composition comprises about 200 mg of Vitamin C (from mineral ascorbates); about 2000 IU of Vitamin D3 (as cholecalciferol); about 50 mg of Vitamin B1(as thiamin Hcl); about 25 mg of Vitamin B2 (as riboflavin); about 50 mg of Niacin (as niacinimide); about 50 mg of Pantothenic acid (as d-calcium pantothenate); about 50 mg of Vitamin B6 (as pyridoxine HCl); about 420 mcg of Folic acid; about 250 mcg of Vitamin B12 (as methylcobalamine); about 100 mg of Calcium (as calcium carbonate); about 16 mg of Zinc (as zinc ascorbate); about 55 mg of Magnesium (as magnesium ascorbate); about 500 mg of Acetyl-L-Carnitine; about 150 mg of DMAE complex (standardized at 37% DMAE equal to 55 mg); about 125 mg of PS complex (standardized at 20% PS equal to 25 mg); about 50 mg of a Proprietary blend (including bacopa monnieri (root) extract, gingko biloba (leaf) extract, passion (flower), lemon balm (leaf), and ashwagandha (root) as an exemplary blend); about 25 mg of an Organic freeze dried fruit juice blend (including concord grape, red raspberry, pineapple, cranberry, acai, pomegranate, acerola cherry, bilberry, lingonberry, black currant, aronia, sour cherry, and black raspberry as an exemplary blend); and about 25 mg of an Organic freeze dried greens blend (including barley (grass), broccoli (whole), beet (root), carrot (root), alfalfa (root), and oat (grass) as an exemplary blend).

The aforementioned exemplary composition contains natural ingredients and is casein free, gluten free, yeast free, as well as having no additives, flavoring, or preservatives that are artificial. The nutrition supplement just described supports healthy neural development, cognitive function, concentration, memory, alertness, learning, and mood and behavioral stability which are all are rooted in biological processes that are supported by the essential nutrients provided by the current invention.

The vitamin composition of the current invention may be administered with as a dietary supplement or through a pharmaceutically acceptable carrier. A pharmaceutically acceptable carrier may be any compatible, non-toxic, non-antioxidant substance suitable to deliver the components. The supplement may contain other pharmaceutically acceptable substances as required to approximate physiological conditions such as a pH adjusting and buffering agent, disbursing agents, toxicity adjusting agents, flavoring agents and like. The concentration of the components in these formulations may vary and will be selected primarily on the particular dosage and mode of administration selected. Methods for preparing supplements are well-known or will be apparent to those skilled in the art and are described in more detail in, for example, Remington's Pharmaceutical Science, 15th ed., Mack Publishing Company, Easton, Pa., incorporated by reference herein, or as understood in the U.S., Pharmacopeia/National Formulary (2009).

The supplement is useful for oral administration. The supplement may be formulated in a variety of dosage forms, such as powders, tablets, capsules, oral solutions, chewable tablets, or suspensions.

Preferably, the supplement is administered orally. For oral administration, solid or fluid dosage forms can be prepared. For preparing solid compositions such as powders or tablets, the components are mixed with conventional ingredients, such as talc, vegetable stearic acid, vegetable magnesium stearate, silica, guar gum, sorbitol, xylitol, erythritol, lecithin (from soy), stevia, citric acid, maltodextrin, beta carotene, natural coloring, natural flavoring, cocoa powder and functionally similar materials, as pharmaceutical carriers. Capsules are prepared by mixing the components with an inert pharmaceutical diluent and filling the mixture into a hard gelatin capsule. Soft gelatin capsules are prepared by machine encapsulation of a slurry of the components with an acceptable vegetable oil, light liquid petrolatum or other inert oil. Fluid unit dosage forms for oil administration such as serum and suspensions can be prepared. The components may be dissolved in an aqueous vehicle together with sugar, sweetening and flavoring agents and preservatives to form a serum. Suspensions can be prepared with an aqueous vehicle and a disbursing agent such as acacia, tragacanth, methylcellulose and the like. In accordance with the invention, any carrier, filler or other substance associated with the components of the invention used to prepare a tablet, capsule will be prepared in accordance with methods familiar to those skilled in the art.

### EXAMPLES

By way of non-limiting example, a supplement formulation containing the following is disclosed.

| | Weight range |
|---|---|
| Ingredients | (in mg) |
| Vitamin C (from mineral ascorbates) | 1-600 |
| Vitamin D3 (as cholecalciferol) | .001-.5 |
| Thiamin (B1) (as thiamin HCI) | 1-100 |
| Riboflavin (B2) | 1-50 |
| Niacin (as niacinamide) | 1-100 |
| Vitamin B6 (as pyridoxine HCI) | 1-100 |
| Folic acid | .05-5 |
| Vitamin B12 (as methylcobalamine) | .001-1 |
| Pantothenic acid (as d-calcium pantothenate) | 1-100 |
| Calcium (as calcium carbonate) | 1-500 |
| Magnesium (as magnesium ascorbate) | 1-500 |
| Zinc (as zinc ascorbate) | 1-35 |
| Chromium (as chromium picolinate), | .001-1 |
| Sugar (as glucose) | 250-15000 |
| Protein (from pea protein isolate) | 50-10000 |
| Acetyl-L-Carnitine | 1-5000 |
| DMAE complex | 1-5000 |
| PS complex | 1-5000 |
| Proprietary blends | 1-10000 |
| (L-Glutamine, N-Acetyl-L-Tyrosine, L- | |
| Phenylalanine, Taurine, Methionine, Valine, | |
| Isoleucine, 5 HTP (Hydroxytryptophan), L-Taurine, | |
| N-Acetyl-Tyrosine, N-Acetyl-L-Cysteine, Alpha | |
| Lipoic Acid, Alpha GPC | |
| (Glycerylphosphoricholine) complex, Bacopa | |
| Monnieri (root) extract, Gingko Biloba (leaf) | |
| extract, Passion (flower), Lemon Balm (leaf), Gotu | |
| Kola (root), Ashwagandha (root), Choline Bitartrate | |
| complex, Panax Ginseng (root) extract, Turmeric) | |
| Organic freeze dried fruit juice blends | 1-500 |
| (concord grape, red raspberry, pineapple, cranberry, | |
| acai, pomegranate, acerola cherry, bilberry, | |
| lingonberry, black currant, aronia, sour cherry, | |
| black raspberry) | |
| Organic freeze dried greens blends | 1-500 |
| (barley (grass), broccoli (whole), beet (root), carrot | |
| (root), alfalfa (root), oat (grass)) | |
| Protein digestive enzyme blends | 1-250 |
| (Protease 4.5, peptidase, bromelain, protease 6.0, | |
| protease 3.0, L planatrum, B bifidum) | |

In yet another non-limiting example a supplement formulation containing the following is disclosed.

| | Weight range |
|---|---|
| Ingredients | (in mg) |
| Vitamin C (from mineral ascorbates) | 200 |
| Vitamin D3 (as cholecalciferol) | .05 |
| Thiamin (B1) (as thiamin HCI) | 50 |
| Riboflavin (B2) | 25 |
| Niacin (as niacinamide) | 50 |
| Vitamin B6 (as pyridoxine HCI) | 50 |
| Folic acid | .42 |
| Vitamin B12 (as methylcobalamine) | .25 |
| Pantothenic acid (as d-calcium pantothenate) | 50 |
| Calcium (as calcium carbonate) | 100 |
| Magnesium (as magnesium ascorbate) | 55 |
| Zinc (as zinc ascorbate) | 16 |
| Sugar (as glucose) | 8000 |
| Acetyl-L-Carnitine | 500 |
| DMAE complex | 150 |
| (standardized at 37% DMAE equal to 55 mg) | |
| PS complex | 125 |
| (standardized at 20% PS equal to 25 mg) | |
| Proprietary blends | 50 |
| (Bacopa Monnieri (root) extract, Gingko Biloba | |
| (leaf) extract, Passion (flower), Lemon Balm (leaf), | |
| Gotu Kola (root), Ashwagandha (root)) | |
| Organic freeze dried fruit juice blends | 25 |
| (concord grape, red raspberry, pineapple, cranberry, | |
| acai, pomegranate, acerola cherry, bilberry, | |
| lingonberry, black currant, aronia, sour cherry, | |
| black raspberry) | |
| Organic freeze dried greens blends | 25 |
| (barley (grass), broccoli (whole), beet (root), carrot | |
| (root), alfalfa (root), oat (grass)) | |
| Protein digestive enzyme blends | |
| (Protease 4.5, peptidase, bromelain, protease 6.0, | |
| protease 3.0, L planatrum, B bifidum) | |

It will be understood by those skilled in the art, that various components of the aforementioned examples can be substituted with similar therapeutic components. For example, preferably, the pharmaceutically acceptable magnesium compound is magnesium oxide. By way of further example, pharmaceutically acceptable zinc compounds include, but are not limited to, zinc sulfate, zinc chloride, zinc oxide, and combinations thereof. Preferably, the pharmaceutically acceptable zinc compound of the current invention is zinc oxide.

While various embodiments of the supplement have been disclosed, it should be understood that modifications and adaptations thereof will occur to one skilled in the art. Other features and aspects of this invention will be appreciated by those skilled in the art upon reading and comprehending this disclosure. Such features, aspects, and expected variations and modifications of the reported results and examples are clearly within the scope of the invention.

1) A nutritional supplement composition, comprising a therapeutically effective amount of Vitamin C, Vitamin D3, Thiamin (B1), Riboflavin (B2), Niacin, Vitamin B6, Folic acid, Vitamin B12, Pantothenic acid, Calcium, Magnesium, Zinc, Chromium, Sugar, Protein, Acetyl-L-Carnitine, DMAE (Dimethylaminoethanol) complex, PS (Phosphatidylserine) complex, Proprietary blends, Organic freeze dried fruit juice blends, Organic freeze dried greens blends, and Protein digestive enzyme blends.

2) The nutritional supplement composition of claim 1, wherein said Vitamin C is in the form of mineral ascorbates; said Vitamin D3 is in the form of cholecalciferol; said Thiamin (B1) is in the form of thiamin HCl; said Niacin is in the form of niacinamide; said Vitamin B6 is in the form of pyridoxine HCl; said Vitamin B 12 is in the form of methylcobalamine; said Pantothenic acid is in the form of d-calcium pantothenate; said Calcium is in the form of calcium carbonate; said Magnesium is in the form of magnesium ascorbate; said Zinc is in the form of zinc ascorbate; said Chromium is in the form of chromium picolinate; said Sugar is in the form of glucose; said Protein is in the form of pea protein isolate; said Proprietary blends consisting essentially of L-Glutamine, N-Acetyl-L-Tyro sine, L-Phenylalanine, Taurine, Methionine, Valine, Isoleucine, 5 HTP (Hydroxytryptophan), L-Taurine, N-Acetyl-Tyrosine, N-Acetyl-L-Cysteine, Alpha Lipoic Acid, Alpha GPC (Glycerylphosphoricholine) complex, Bacopa Monnieri (root) extract, Gingko Biloba (leaf) extract, Passion (flower), Lemon Balm (leaf), Gotu Kola (root), Ashwagandha (root), Choline Bitartrate complex, Panax Ginseng (root) extract, and Turmeric; said Organic freeze dried fruit juice blends consisting essentially of concord grape, red raspberry, pineapple, cranberry, acai, pomegranate, acerola cherry, bilberry, lingonberry, black currant, aronia, sour cherry, black raspberry; said Organic freeze dried greens blends consisting essentially of barley (grass), broccoli (whole), beet (root), carrot (root), alfalfa (root), oat (grass); and said Protein digestive enzyme blends consisting essentially of Protease 4.5, peptidase, bromelain, protease 6.0, protease 3.0, L planatrum, B bifidum.

3) The nutritional supplement composition of claim 1, where said composition comprises about 200 mg of Vitamin C, about 2000 IU of Vitamin D3, about 50 mg of Thiamin (B1), about 25 mg of Riboflavin (B2), about 50 mg of Niacin, about 50 mg of Vitamin B6, about 0.420 mg of Folic acid, about 0.250 mg of Vitamin B12, about 50 mg of Pantothenic acid, about 100 mg of Calcium, about 55 mg of Magnesium, about 16 mg of Zinc, about 8000 mg of Sugar, about 500 mg of Acetyl-L-Carnitine, about 150 mg of DMAE (Dimethylaminoethanol) complex, about 125 mg of PS (Phosphatidylserine) complex, about 50 mg of Proprietary blend, about 25 mg of Organic freeze dried fruit juice blends, and about 30 mg of Organic freeze dried greens blends.

4) The nutritional supplement composition of claim 1, where said composition comprises about 200 mg of Vitamin C, about 1000 IU of Vitamin D3, about 7 mg of Thiamin (B1), about 8 mg of Riboflavin (B2), about 21 mg of Niacin, about 10 mg of Vitamin B6, about 0.420 mg of Folic acid, about 0.030 mg of Vitamin B12, about 15 mg of Pantothenic acid, about 100 mg of Calcium, about 50 mg of Magnesium, about 8 mg of Zinc, about 7000 mg of Sugar, about 40 mg of Acetyl-L-Carnitine, about 125 mg of DMAE (Dimethylaminoethanol) complex, about 125 mg of PS (Phosphatidylserine) complex, about 55 mg of Proprietary blend, about 25 mg of Organic freeze dried fruit juice blends, and about 30 mg of Organic freeze dried greens blends.

5) The nutritional supplement composition of claim 2, where said composition comprises about 200 mg of Vitamin C, about 2000 IU of Vitamin D3, about 50 mg of Thiamin (B1), about 25 mg of Riboflavin (B2), about 50 mg of Niacin, about 50 mg of Vitamin B6, about 0.420 mg of Folic acid, about 0.250 mg of Vitamin B12, about 50 mg of Pantothenic acid, about 100 mg of Calcium, about 55 mg of Magnesium, about 16 mg of Zinc, about 8000 mg of Sugar, about 500 mg of Acetyl-L-Carnitine, about 150 mg of DMAE (Dimethylaminoethanol) complex, about 125 mg of PS (Phosphatidylserine) complex, about 50 mg of Proprietary blend, about 25 mg of Organic freeze dried fruit juice blends, and about 30 mg of Organic freeze dried greens blends.

6) The nutritional supplement composition of claim 2, wherein said composition comprises about 200 mg of Vitamin C, about 1000 IU of Vitamin D3, about 7 mg of Thiamin (B1), about 8 mg of Riboflavin (B2), about 21 mg of Niacin, about 10 mg of Vitamin B6, about 0.420 mg of Folic acid, about 0.030 mg of Vitamin B12, about 15 mg of Pantothenic acid, about 100 mg of Calcium, about 50 mg of Magnesium, about 8 mg of Zinc, about 7000 mg of Sugar, about 40 mg of Acetyl-L-Carnitine, about 125 mg of DMAE (Dimethylaminoethanol) complex, about 125 mg of PS (Phosphatidylserine) complex, about 55 mg of Proprietary blend, about 25 mg of Organic freeze dried fruit juice blends, and about 30 mg of Organic freeze dried greens blends.

7) The nutritional supplement composition of claim 1, wherein said composition comprises between 1-600 mg of Vitamin C, between 0.001-0.5 mg of Vitamin D3, between 1-100 mg of Thiamin (B1), between 1-50 mg of Riboflavin (B2), between 1-100 mg of Niacin, between 1-100 mg of Vitamin B6, between 0.05-5 mg of Folic acid, between 0.001-1 mg of Vitamin B12, between 1-100 mg of Pantothenic acid, between 1-500 mg of Calcium, between 1-500 mg of Magnesium, between 1-35 mg of Zinc, between 0.001-1 mg of Chromium, between 250-15000 of Sugar, between 50-10000 mg of Protein, between 1-5000 mg of Acetyl-L-Carnitine, between 1-5000 mg of DMAE (Dimethylaminoethanol) complex, between 1-5000 mg of PS (Phosphatidylserine) complex, between 1-10000 mg of Proprietary blends, between 1-500 mg of Organic freeze dried fruit juice blends, between 1-500 mg of Organic freeze dried greens blends, and between 1-250 mg of Protein digestive enzyme blends.

8) The nutritional supplement composition of claim 5, wherein said composition is a chewable tablet.

9) The nutritional supplement composition of claim 6, wherein said composition is a chewable tablet.

10) The nutritional supplement composition of claim 5, wherein said composition is a powder.

11) The nutritional supplement composition of claim 6, wherein said composition is a powder.

12) The nutritional supplement composition of claim 5, wherein said composition is formulated into a chewable form suitable for human consumption.

13) The nutritional supplement composition of claim 6, wherein said composition is formulated into a chewable form suitable for human consumption.

14) The nutritional supplement composition of claim 10, wherein said powder is put into a solution suitable for dissolving said powder.

15) The nutritional supplement composition of claim 11, wherein said powder is put into a solution suitable for dissolving said powder.

16) A dietary supplement system, suitable to address vitamin, mineral and essential amino acid requirements of humans required to support brain function, cognitive function, behavior and mood of a human, said system comprising a once daily delivery of a composition comprising a therapeutically effective amount of Vitamin C (from mineral ascorbates), Vitamin D3 (as cholecalciferol), Thiamin (B1) (as thiamin HCl), Riboflavin (B2), Niacin (as niacinamide), Vitamin B6 (as pyridoxine HCl), Folic acid, Vitamin B 12 (as methylcobalamine), Pantothenic acid (as d-calcium pantothenate), Calcium (as calcium carbonate), Magnesium (as magnesium ascorbate), Zinc (as zinc ascorbate), Chromium (as chromium picolinate), Sugar (as glucose), Protein (from pea protein isolate), Acetyl-L-Carnitine, DMAE (Dimethylaminoethanol) complex, PS (Phosphatidylserine) complex, Proprietary blends (L-Glutamine, N-Acetyl-L-Tyrosine, L-Phenylalanine, Taurine, Methionine, Valine, Isoleucine, 5 HTP (Hydroxytryptophan), L-Taurine, N-Acetyl-Tyrosine, N-Acetyl-L-Cysteine, Alpha Lipoic Acid, Alpha GPC (Glycerylphosphoricholine) complex, Bacopa Monnieri (root) extract, Gingko Biloba (leaf) extract, Passion (flower), Lemon Balm (leaf), Gotu Kola (root), Ashwagandha (root), Choline Bitartrate complex, Panax Ginseng (root) extract, Turmeric), Organic freeze dried fruit juice blends (concord grape, red raspberry, pineapple, cranberry, acai, pomegranate, acerola cherry, bilberry, lingonberry, black currant, aronia, sour cherry, black raspberry), Organic freeze dried greens blends (barley (grass), broccoli (whole), beet (root), carrot (root), alfalfa (root), oat (grass)), and Protein digestive enzyme blends (Protease 4.5, peptidase, bromelain, protease 6.0, protease 3.0, L planatrum, B bifidum).
